(19) United States
(12) Patent Application Publication
(10) Pub. No.: US 2005/0261705 A1
(43) Pub. Date: Nov. 24, 2005

(54) Device to remove kidney stones

(76) Inventor: Christopher W. Gist, Flowood, MS (US)

Correspondence Address: LITMAN LAW OFFICES, LTD, PO BOX 15035, CRYSTAL CITY STATION, ARLINGTON, VA, 22215, US

(21) Appl. No.: 10/849,887

(22) Filed: May 21, 2004

(51) Int. Cl. A61B 17/26

(52) U.S. Cl. 606/113

(57) ABSTRACT

The device to remove kidney stones is a medical retrieval basket device adapted to provide irrigation and suction to the region of the basket. The device includes a retrieval basket assembly having a sheath, an elongated member extending through the sheath, and a basket assembly coupled to the distal end of the elongated member, in a generally known arrangement. The retrieval basket assembly extends from a handle which provides suction and irrigation to, and through, the retrieval basket assembly. Fittings on the handle connect fluid and suction sources through fluid conduits within the handle to the sheath of the retrieval basket assembly. In one embodiment, the retrieval basket assembly's sheath has an expandable distal end portion that improves the retrieval basket assembly's ability to capture debris from lithotripsy of a stone.

### BACKGROUND OF THE INVENTION

1. Field of the Invention

The present invention relates to medical devices for retrieving objects from within a body. More particularly, the invention is a device to remove kidney stones that includes means for providing irrigation and suction.

2. Description of the Related Art

Medical retrieval devices are used to remove biological materials, such as kidney stones, from a body. A common use of these devices is the removal of stones from a patient's urinary tract or kidneys. A typical medical retrieval device includes a basket that extends from, or may be withdrawn into, an elongated sheath. The basket is typically disposed on the end of an elongated member, such as a wire, catheter, or the like, running lengthwise through a lumen within the sheath. The basket is operated between its extended and withdrawn positions by manipulating the elongated member within the sheath, or by manipulating the sheath over the elongated member. A handle is often provided to support the sheath, and includes an actuating mechanism such as a lever or slide member operatively connected to the sheath or elongated member to extend and retract the basket.

U.S. Pat. No. 6,626,915, issued on Sep. 30, 2003 to R. Leveillee, discloses a medical retrieval device generally of the type discussed above.

U.S. Pat. No. 6,695,834, issued on Feb. 24, 2004 to B. Gellman, discloses an apparatus for stone removal from a body. The apparatus is similar to the type discussed above, having a basket disposed on a distal end of an elongated member, the elongated member being disposed within the lumen of a sheath. The elongated member, in some embodiments, may itself have a lumen extending its length, and the lumen may be sized to accommodate within the elongated member a lithotriptor, such as a fiber optic fiber in communication with a laser, or to serve as a conduit for fluids such as drugs or normal saline solution for irrigation.

U.S. Pat. No. 6,398,791, issued on Jun. 4, 2002 to L. Que et al., discloses, in a medical device having a handle, a sheath, and an object-engaging unit, a sheath having a flexible portion near the distal end of the sheath. The flexible portion of the sheath allows for improved manipulation of the sheath through twisting and turning body tracts, while retaining rigidity at the distal end of the sheath to prevent the sheath from deforming while maneuvering the sheath or when the object-engaging unit is retracted into the sheath.

U.S. Patent Application Publication 2002/0068943, published on Jun. 6, 2002, discloses a laser-resistant medical retrieval device. U.S. Pat. No. 6,500,182, issued on Dec. 31, 2002 to T. Foster, discloses a medical retrieval device generally of the type discussed above, describing several different basket styles.

Retrieval devices such as those described above are often used in conjunction with an endoscope. The sheath of the retrieval device is routed through a lumen within the endoscope, allowing a physician to manipulate the retrieval device with the aid of the image provided by the endoscope.

U.S. Pat. No. 5,199,417, issued on Apr. 6, 1993 to R. Muller et al., discloses an endoscope having working channels through which a working tool may be passed for performing a procedure within a body cavity or passageway.

When performing an endoscopic procedure with a retrieval device within a body cavity or passageway, and especially while performing lithotripsy using a lithotriptor, a common problem is that debris from the lithotripsy clouds the endoscope's field of view. A means for clearing such debris is desirable both to remove the debris as the lithotripsy progresses, and to maintain a clear field of view through the endoscope during the procedure.

U.S. Patent Application Publication 2002/0103477, published on Aug. 1, 2002, discloses a laser lithotripsy device with suction. A suction conduit runs alongside a laser energy conduit, the laser energy conduit directing laser energy to a region proximate to the tip of the suction conduit. Suction applied to the suction conduit retains stones and stone fragments in the region of the laser energy, and removed from the stones and fragments by the laser are removed from the field by the suction.

U.S. Pat. No. 6,517,531, issued on Feb. 11, 2003 to C. Liu et al., discloses a medical suction device for fragmenting objects and aspirating remaining debris. An elongated member, having dual lumens, extends from a handle. A first lumen provides a suction passageway, while a second lumen receives a laser fiber. Suction is delivered to the first lumen through the handle from a suction connector used to connect the device with an external suction pump. A pushbutton disposed within the handle allows the suction to be selectively applied or disabled. Substituting the suction pump for a pump that provides bidirectional flow, irrigation can be supplied to the device through the suction connector. While the ability to provide both suction and irrigation is useful, the need to operate a separate pump to reverse its flow may require, at least for a moment, the physician to turn his attention away from the procedure and to the supporting equipment.

U.S. Pat. No. 6,264,664, issued on Jul. 24, 2001 to F. Avellanet, discloses a surgical basket instrument having a sheath, a shaft extending through the sheath, and a basket assembly coupled to the distal end of the shaft. A hollow channel in the shaft receives at least one fiber optic attachable to a laser for destroying calculi. In one embodiment, a fluid source is coupled to the channel. The illustrated fluid source is a syringe for manually injecting fluid into the channel, which carries the risk of applying excessive fluid and flushing a stone or debris away from the distal end of the device and further into a body cavity or passageway.

None of the above inventions and patents, taken either singly or in combination, is seen to describe the instant invention as claimed. Thus a device to remove kidney stones solving the aforementioned problems is desired.

### SUMMARY OF THE INVENTION

The device to remove kidney stones is a medical retrieval basket device adapted to provide irrigation and suction to the region of the basket. The device includes a retrieval basket assembly comprising a sheath, an elongated member extending through the sheath, and a basket assembly coupled to the distal end of the elongated member, in a generally known arrangement. The retrieval basket assembly extends from a handle that is adapted to provide suction and irrigation to, and through, the retrieval basket assembly.

The handle has a fitting for the connection of a fluid source and a fitting for the connection of a suction source. Internal plumbing connects the fluid and suction sources to the sheath of the retrieval basket assembly. A separate valve for each of the fluid and suction sources allows each to be independently activated or deactivated. Practically speaking, however, since the fluid source and suction source are commonly joined to the retrieval basket assembly's sheath, irrigation or suction may be applied separately, but not simultaneously.

The handle also includes a fiber optic port to allow the insertion of a fiber optic into the handle and through the retrieval basket assembly. The fiber optic port is adapted to provide a fluid-tight seal around the fiber optic, when the fiber optic is inserted. Additionally, the fiber optic port provides a fluid tight seal when the fiber optic is removed.

In one embodiment, the retrieval basket assembly's sheath has an expandable distal end portion that improves the retrieval basket assembly's ability to capture debris from lithotripsy of a stone.

These and other features of the present invention will become readily apparent upon further review of the following specification and drawings.

### BRIEF DESCRIPTION OF THE DRAWINGS

FIG. 1 is a side elevational view of a device to remove kidney stones according to the present invention.

FIG. 2A is a diagrammatic view in section of a handle for a device to remove kidney stones according to the present invention.

FIG. 2B is a diagrammatic view in section of a handle for a device to remove kidney stones according to the present invention, shown with a fiber optic extending through the handle.

FIG. 2C is a diagrammatic view in section of a first alternate embodiment of a handle for a device to remove kidney stones according to the present invention.

FIG. 2D is a diagrammatic view in section of a second alternate embodiment of a handle for a device to remove kidney stones according to the present invention, the handle containing an electrical irrigation pump.

FIG. 3 is a side elevational view of a device to remove kidney stones according to the present invention, having a sheath with an expandable distal end portion.

FIG. 4A is diagrammatic view of a clinical application of an embodiment of the device illustrated in FIG. 1, with the basket extended to capture an object.

FIG. 4B is diagrammatic view of a clinical application of an embodiment of the device illustrated in FIG. 1, with the basket containing a captured object withdrawn into the expandable end portion of the sheath.

Similar reference characters denote corresponding features consistently throughout the attached drawings.

### DETAILED DESCRIPTION OF THE PREFERRED EMBODIMENT

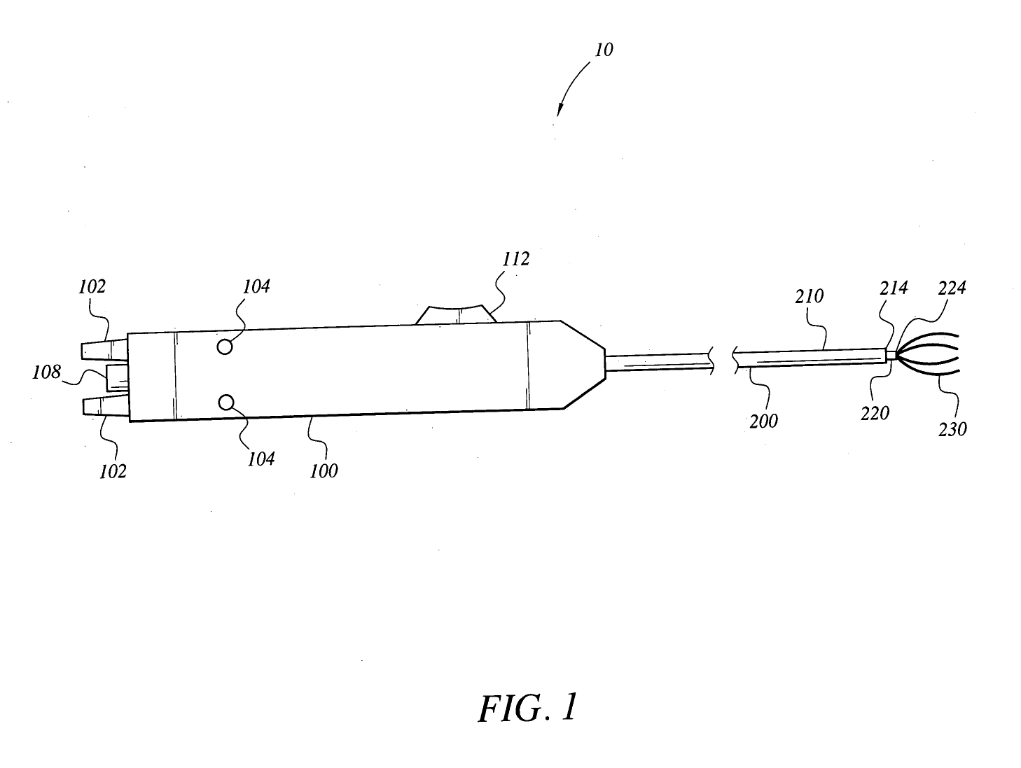

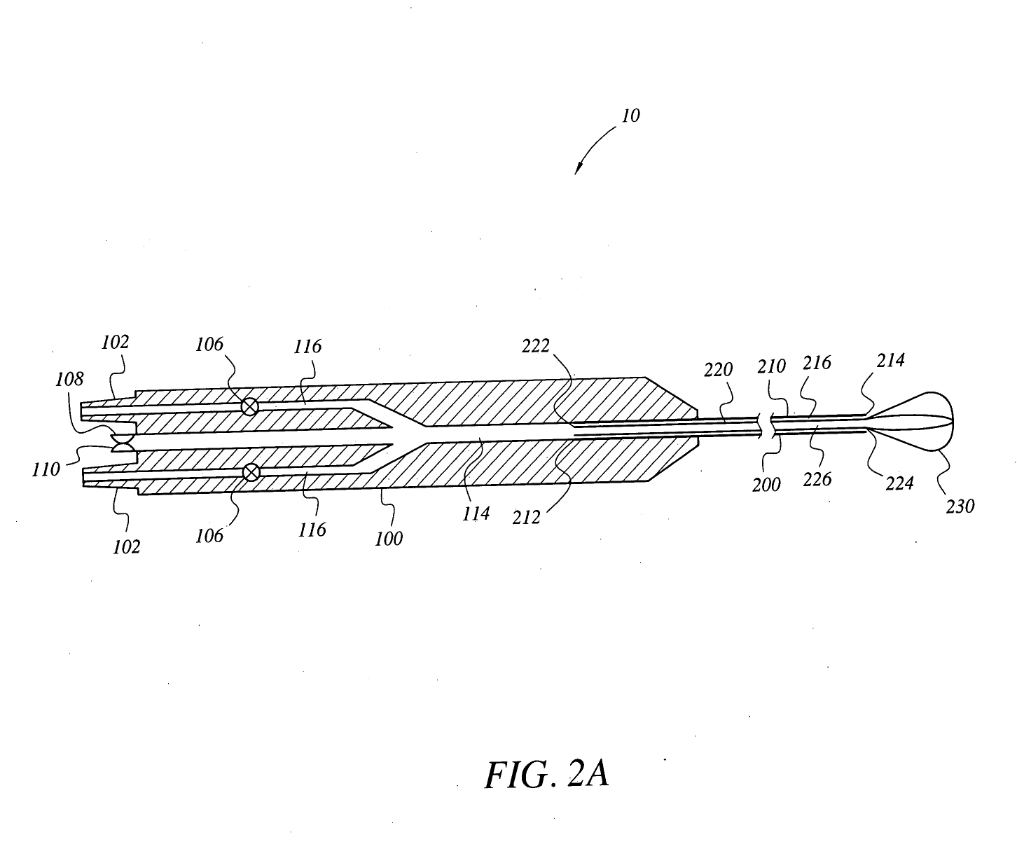

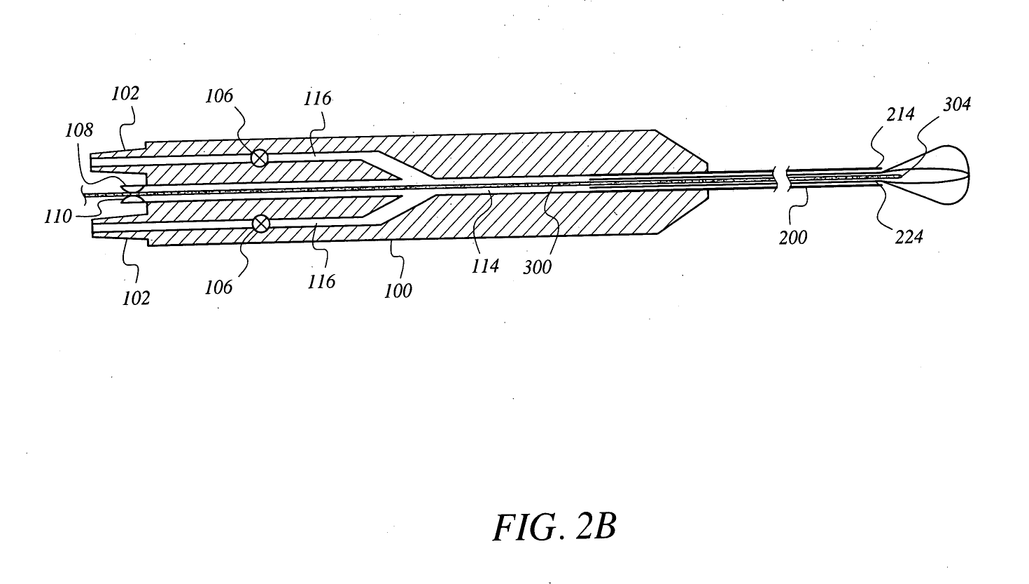

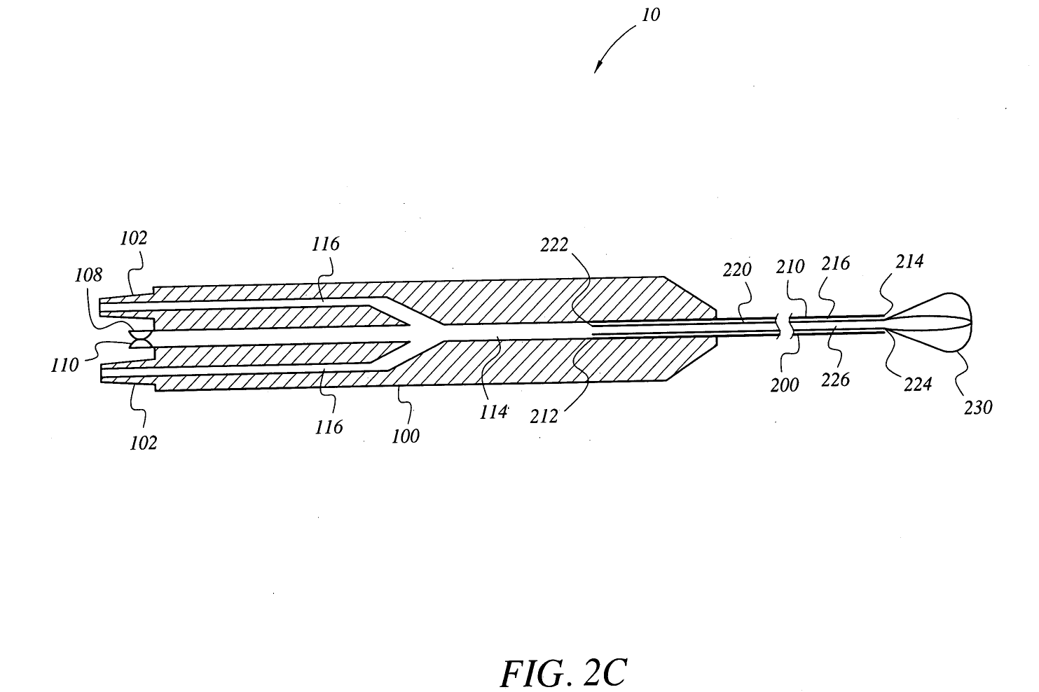

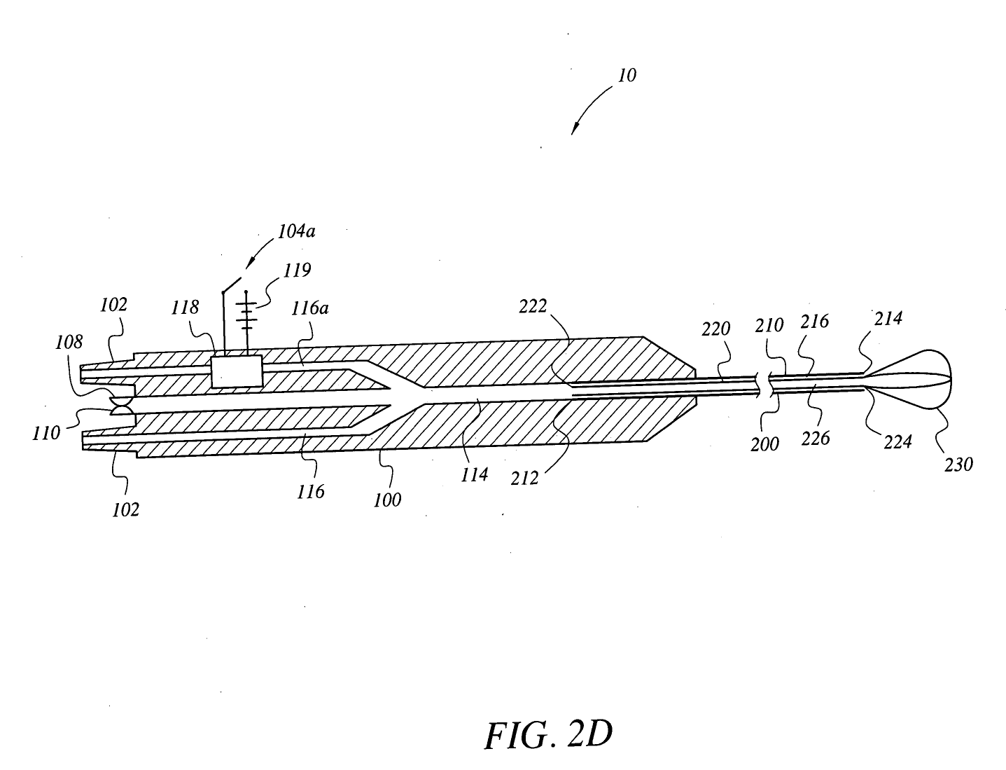

The present invention is a device to remove kidney stones, designated generally as 10 in the drawings. Referring to FIGS. 1, 2A, and 2B, the device to remove kidney stones 10 comprises a retrieval basket assembly 200 and a handle 100, the retrieval basket assembly 200 extending from a distal end of the handle 100, there being a collapsible basket 230 disposed at a distal end of the retrieval basket assembly 200. Internal plumbing in the handle 100 provides irrigation and suction through the retrieval basket assembly 200 to the basket 230 area. Fluid connectors 102 allow a fluid source and a suction source to be connected to the internal plumbing of the handle 100.

The retrieval basket assembly 200 comprises a sheath 210 having a proximal end 212 and a distal end 214. The sheath 210 has at lease one lumen 216 running its length. An elongated member 220 extends within the lumen 216 of the sheath and has a distal end 224. A collapsible basket 230, of a conventional type that is generally known, is attached to the distal end 224 of the elongated member 220. The elongated member 220 may be, for example, a shaft, catheter, cable, coil, or guide wire. In the embodiment illustrated, the elongated member 220 has a lumen 226 that allows for the passage of a fiber optic 300. With the fiber optic 300 removed, fluids pass through the lumen 226 during irrigation or suction operations. Additionally, fluids may pass through the lumen 216 of the sheath 210, around the elongated member 220. In other embodiments, where the elongated member 220 is thin in relation to the lumen 216 of the sheath 210, fluids pass through lumen 216 of the sheath 210 around the elongated member 220.

The internal plumbing of the handle 100 includes a central conduit 114 that extends the length of the handle 100. The proximal end 212 of the sheath 210 of the retrieval basket assembly 200 is attached to a distal end of the handle 100 in fluid communication with the central conduit 114. A fiber optic port 108 is disposed on a proximal end of the handle 100 in communication with the central conduit 114. The fiber optic port 108 includes a seal 110 that allows a fiber optic 300 to be inserted through the port 108 and into the handle 100, the fiber optic 300 extending through the handle 100 and through the basket assembly 200. The seal 110 provides a fluid-tight seal around the fiber optic 300, and forms a fluid tight seal when the fiber optic 300 is removed. In use, the fiber optic 300 is inserted through the fiber optic port 108 and extended until a distal end 304 of the fiber optic 300 emerges from the distal end of the elongated member 220, where the fiber optic 300 is used to apply laser impulses to a stone captured in the region of the basket 230. Removal of the fiber optic 300 facilitates application of irrigation and suction through the retrieval basket assembly 200.

Fluid conduits 116 join fluid connectors 102, extending from the handle 100, in fluid communication with the central conduit 114. Each of the fluid conduits 116 includes an inline valve 106, operable by a pushbutton 104 or other mechanism located on the handle 100, so that a fluid or suction source in connection with the fluid conduit 100 may be selectively applied or disabled. Providing the handle 100 with a pair of fluid conduits 116 allows for one of the fluid conduits 116 to be connected to a fluid source, while the other is connected to a suction source, thereby allowing irrigation and suction to be selectively applied to the retrieval basket assembly 200. Additional fluid conduits (not shown) may be provided to allow delivery of additional fluids, such as medications, through the retrieval basket assembly 200.

The handle 100 contains an operating mechanism 112, which is operatively connected to the sheath 210. Moving the operating mechanism 112 into a first position causes the sheath 210 to extend over the elongated member 220 and over the basket 230, thereby collapsing the basket 230 within the sheath 210. Moving the operating mechanism 112 into a second position causes the sheath 210 to retract from the basket 230, thereby extending the basket 230 from the sheath 210 and allowing the basket to expand beyond the distal end 214 of the sheath 210. Alternatively, the operating mechanism 112 can be operatively connected to the elongated member 220, whereby moving the operating mechanism 112 causes the basket to move in and out of the sheath 210 between a collapsed position within the sheath 210 and an expanded position extended from the distal end 214 of the sheath 210.

In a first alternate embodiment, shown in FIG. 2C, valves are omitted from the fluid conduits 116, the fluid conduits 116 passing uninterrupted between the fluid connectors 102 and the central conduit 114. In this embodiment, the pushbuttons 104 on the handle 100 are electrical switches for electrical control of external electrically actuated pump mechanisms.

In a second alternate embodiment, illustrated in FIG. 2D, an electrically operated irrigation pump 118 is contained within the handle 100, the pump 118 being inline in one of the fluid conduits 116a, pushbutton 104a being an electrical switch in electrical communication with the pump 118 and with a power supply 119. The power supply 119 may be a battery or other power source external to or contained within the handle 100, or may be an electrical connector adapted for connection with an external power source.

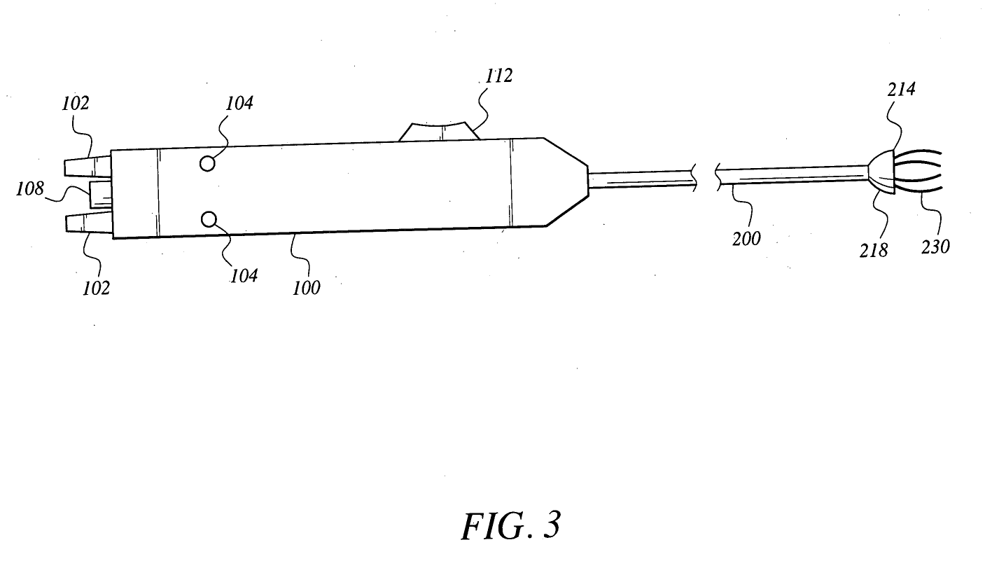

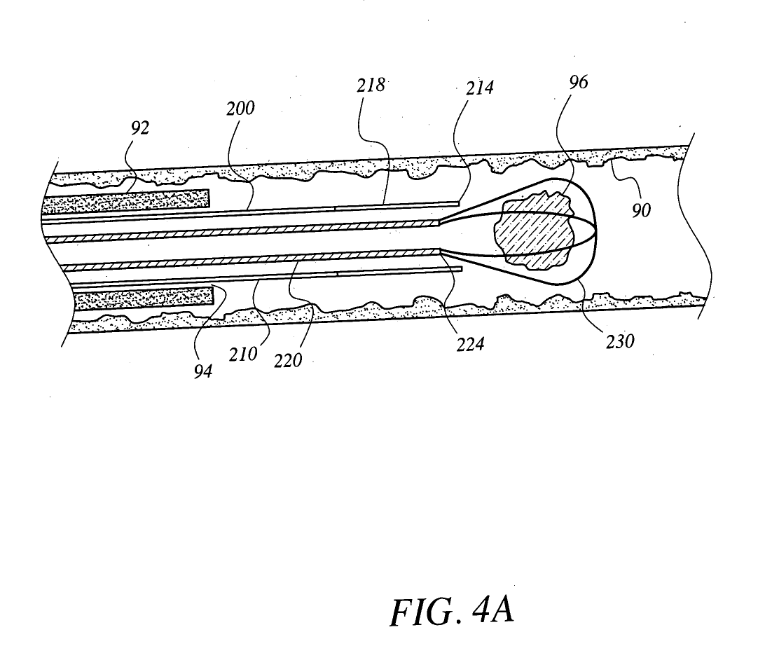

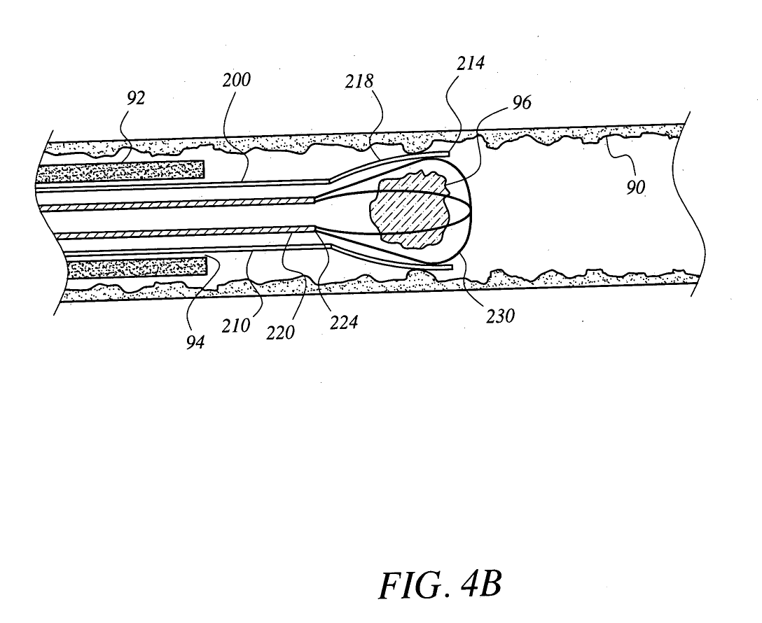

Turning now to FIGS. 3, 4A, and 4B, an embodiment of the device to remove kidney stones 10 is illustrated wherein the sheath 210 has a short, expandable portion 218 at the distal end 214 of the sheath 210. The expandable portion 218 of the sheath 210 is made of an elastic material so that the expandable portion 218 expands as the basket 230 is withdrawn into the distal end 214 of the sheath 210.

Referring particularly to FIGS. 4A and 4B, the end portion of the retrieval basket assembly 200 is shown inserted through a lumen or working channel 94 of an endoscope or ureteroscope 92 into a body passageway 90, such as a ureter. In FIG. 4A, the retrieval basket assembly 200 is shown positioned for the basket 230 to capture a stone 96 within a body passageway 90. FIG. 4B shows the basket 230 with the stone 96 captured within the basket 230, the basket 230 being withdrawn to expand the expandable portion 218 of the sheath 210. With the expandable portion 218 of the sheath 210 expanded, the expandable portion 218 creates a restriction within the passageway 90, serving to facilitate in the collection by the retrieval basket assembly 200 of the debris created by lithotripsy of the stone 96. Thus, when suction is applied through the retrieval basket assembly 200, the expandable portion 218 of the sheath 210 helps to direct debris from the stone 96 toward the distal end 214 of the sheath 210 and the distal end 224 of the elongated member 220 where the debris is drawn away by suction applied from the handle 100.

It is to be understood that the present invention is not limited to the embodiment described above, but encompasses any and all embodiments within the scope of the following claims.

1. A device to remove kidney stones, comprising:
a handle having a distal end and a proximal end, the handle having a central conduit extending therethrough;
a fiber optic port disposed on the proximal end of said handle, the fiber optic port being in communication with said central conduit;
first and second fluid conduits disposed within said handle, each of the fluid conduits being in fluid communication with said central conduit;
a retrieval basket assembly extending from the distal end of said handle, the retrieval basket assembly having at least one lumen in fluid communication with said central conduit.

2. The device to remove kidney stones according to claim 1, further comprising first and second fluid connectors disposed on said handle, the first and second fluid connectors being in fluid communication with said first and second fluid conduits, respectively.

3. The device to remove kidney stones according to claim 1, further comprising first and second fluid valves disposed inline in said first and second fluid conduits, respectively.

4. The device to remove kidney stones according to claim 1, further comprising an electrically operated fluid pump disposed inline in one of said fluid conduits.

5. The device to remove kidney stones according to claim 4, further comprising an electrical switch in electrical communication with said fluid pump.

6. The device to remove kidney stones according to claim 4, further comprising a power source in electrical communication with said fluid pump.

7. The device to remove kidney stones according to claim 1, wherein said retrieval basket assembly comprises a sheath having at least one lumen, the sheath extending from said handle, the at least one lumen being in fluid communication with said central conduit.

8. The device to remove kidney stones according to claim 7, wherein said sheath has a distal end, a portion of the sheath at the distal end being expandable.

9. The device to remove kidney stones according to claim 7, wherein said retrieval basket assembly further comprises an elongated member slidably disposed within said sheath, the elongated member having a distal end.

10. The device to remove kidney stones according to claim 9, wherein said retrieval basket assembly further comprises a basket disposed on the distal end of said elongated member.

11. The device to remove kidney stones according to claim 9, wherein said elongated member comprises at least one lumen in fluid communication with said central conduit.

12. A retrieval basket assembly, comprising:
a sheath having a proximal end and a distal end, the sheath having at least one lumen, a portion of the distal end being expandable in diameter;
an elongated member having a proximal end and a distal end, the elongated member being slidably disposed in the lumen of said sheath; and
a basket disposed on the distal end of said elongated member.
